(19) United States
(12) Patent Application Publication
(10) Pub. No.: US 2009/0124959 A1
(43) Pub. Date: May 14, 2009

(54) DEVICE FOR TRANSDERMAL DRUG DELIVERY AND METHOD OF OPERATING SUCH A DEVICE

(75) Inventor: Heiko Pelzer, Erkelenz (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/304,519

(22) PCT Filed: Jun. 4, 2007
(86) PCT No.: PCT/IB2007/052084

§ 371 Date: Dec. 12, 2008

(30) Foreign Application Priority Data
Jun. 14, 2006 (EP) 06115459.7

(51) Int. Cl. A61N 7/00

(52) U.S. Cl. 604/22

(57) ABSTRACT

The present invention relates to a device (11) for transdermal drug delivery to a target area (21), e.g. a patient's skin (12). Furthermore, the invention relates to a method of operating such a device (11). In order to provide a technique for transdermal drug delivery, in which the delivery rate is maintained despite the use of ultrasound, a device (11) is suggested comprising a drug reservoir (15) and an ultrasonic membrane transducer (1) adapted to cooperate with said drug reservoir (15), said ultrasonic membrane transducer (1) comprising at least two transducer elements (2) forming a transducer array (5), each transducer element (2) having a membrane (6), a number of electrodes (7) disposed on a surface of each transducer element (2) and coupled to the membrane (6) for applying an electrical field to flex the membrane (6) in order to generate an ultrasonic signal, and a control unit (4) for separately controlling the application of the electrical field to flex the membrane (6) of each transducer element (2) in such a way that the ultrasonic signals (101, 102, 103, . . . ) emitted by the transducer elements (2) exhibit phase differences resulting in a focusable overall ultrasonic signal (10) of the transducer array (5).

The present invention relates to a device for transdermal drug delivery and to a method of operating such a device.

Transdermal drug delivery devices allow a medicinal compound to be absorbed through the skin layers of the patient and into the patient's blood stream. Such devices are affixed to the patient's skin. Many medicinal compounds are not suitable for administration via known transdermal drug delivery devices since they do not permeate through the skin into the blood stream. It is known to use energy, such as ultrasonic energy (ultrasound), to enhance the transdermal delivery of certain drugs. Ultrasound is sound with a frequency over 20,000 Hz, which is about the upper limit of human hearing. The use of ultrasound to increase the permeability of the skin to drug molecules is also known as “sonophoresis” or “phonophoresis”. While the use of certain ultrasonic frequencies for enhancing delivery of certain drugs in certain applications is known, results in such applications have been largely disappointing. In many cases the drug delivery pathway utilized enabled initial quantities of a drug to permeate the patient's skin, but as longer periods of ultrasound were applied to the same location on the skin the delivery rate dropped off or was reduced to zero. In this specification this effect is referred to as “saturation effect”.

Known methods for using ultrasound to enhance transdermal drug delivery require a clinical ultrasonic delivery setting, i.e. the drug delivery is performed in a physician's office, hospital or clinic. These methods are undesirable because of the need for the patient to visit the clinical setting and remain on a treatment table while the ultrasound treatment is used to deliver the drug.

It is an object of the present invention to provide a technique for transdermal drug delivery, in which the delivery rate is maintained despite the use of ultrasound.

This object is achieved according to the invention by a device for transdermal drug delivery to a target area, e.g. a patient's skin, comprising a drug reservoir and an ultrasonic membrane transducer adapted to cooperate with said drug reservoir, said ultrasonic membrane transducer comprising at least two transducer elements forming a transducer array, each transducer element having a membrane, a number of electrodes disposed on a surface of each transducer element and coupled to the membrane for applying an electrical field to flex the membrane in order to generate an ultrasonic signal, a control unit for separately controlling the application of the electrical field to flex the membrane of each transducer element in such a way that the ultrasonic signals emitted by the transducer elements exhibit phase differences resulting in a focusable overall ultrasonic signal of the transducer array. The object of the invention is furthermore achieved by a method of operating a device for transdermal drug delivery to a target area, e.g. a patient's skin, said device comprising a drug reservoir and an ultrasonic membrane transducer adapted to cooperate with said drug reservoir, said ultrasonic membrane transducer comprising at least two transducer elements forming a transducer array, said method comprising the step of controlling the transducer elements in such a way that the ultrasonic signals emitted by the transducer elements exhibit phase differences resulting in a focusable overall ultrasonic signal of the transducer array.

A core idea of the invention is to avoid the above mentioned saturation effect caused by sonophoresis by changing the conditioning of the target area, e.g. the patient's skin, during the drug delivery period. In other words, in contrast to prior art techniques, where a constant level of ultrasound is provided to the complete target area throughout the complete drug delivery period, according to the invention ultrasound is provided to the target area in a more variable way. This includes e.g. variations in the ultrasonic signal level, variations in the receiving area of the target and variations in the receiving time of certain target areas. Preferably, the changes are provided continuously in such a way that the complete target area receives a predetermined constant quantum of ultrasound. Alternatively, the device according to the invention can be adapted in such a way that defined parts of the target area can receive more ultrasound than other parts of the same target area. Those variations can be achieved by means of a well-defined control approach, according to which specifically defined driving voltages are provided to the actuation electrodes of a piezoelectric membrane transducer. By this means, single elements of the transducer can be activated using a time offset, i.e. shifted in time, such that the ultrasonic signals emitted by the transducer elements exhibit phase differences, which lead to an overall ultrasonic signal, which can be purposefully focused across the target area. Another core idea of the invention is to use the ultrasonic membrane transducer as described above in a device for transdermal drug delivery. This would avoid the need for a clinical setting and would increase the patient's comfort. Such a device can, according to the present invention, be provided in a size adequate for a portable solution, i.e. a portable device for transdermal drug delivery can be provided.

These and other aspects of the invention will be further elaborated on the basis of the following embodiments which are defined in the dependent claims.

According to preferred embodiments of the invention, the ultrasonic membrane transducer can comprise a Piezoelectric Micromachined Ultrasound Transducer (PMUT) or a Capacitive Micromachined Ultrasound Transducer (CMUT). Using a PMUT, the membrane comprises a piezoelectric layer and the membrane is flexed by means of piezoelectric actuation of the piezoelectric layer by applying an electrical field to the piezoelectric layer. Using a CMUT, a first electrode of the transducer element is attached to the membrane and a static second electrode of the transducer element faces the first electrode. Applying a voltage between the first electrode and the second electrode generates an electrostatic force flexing the membrane. The CMUT and the PMUT are well-known concepts in the field of thin film ultrasonic membrane transducers and enable the use of low-cost semiconductor processing to manufacture ultrasound transducer arrays. A combination of piezoelectric and electrostatic actuation may be used.

According to a preferred embodiment of the invention, the control unit of the ultrasonic membrane transducer is adapted to control the application of the electrical field to flex the membrane of each transducer element in such a way that the focus of the overall ultrasonic signal can be varied in time and/or size. In particular, the control unit is adapted to control the applied electrical field such that the focus area of the overall ultrasonic signal can be varied in size and/or such that the position of the focus of the overall ultrasonic signal on the target area can be varied in time. In other words, a schedule of exposure can be implemented, according to which the ultrasound application is adapted in a way suitable to prevent saturation effects. Furthermore, an individual ultrasound application can be established for each patient. The variations mentioned can be achieved solely by appropriate applications of electrical fields to the piezoelectric layers of the transducer elements of the ultrasonic membrane transducer, in other words, by applying appropriate drive voltages to the corresponding actuation electrodes.

The depth of penetration of ultrasonic energy into living soft tissue is inversely proportional to the frequency, thus high frequencies are used to improve drug penetration through the skin by concentrating the effect in the outermost skin layer, the stratum corneum. However, shock waves of low frequency can be used to improve the drug material passing the skin. Thus, the invention suggests a combination of low-frequency ultrasound and pulsed high-frequency ultrasound. According to a preferred embodiment of the invention, the control unit of the ultrasonic membrane transducer is adapted to control the application of the electrical field to the membrane of each transducer element in such a way that the overall ultrasonic signal is delivered in a pulsed mode. The pulse rate is preferably between 20,000 and 100,000 pulses per second. According to the invention, pulsing high-frequency ultrasound is used to excite the transport mechanism through the skin, said transport mechanism being based on low frequency ultrasound. In other words, the different transport mechanisms at high ultrasound frequencies and low ultrasound frequencies are used to enhance the permeation of drug particles through the skin. It is believed that the enhanced permeation is based on the occurrence of cavitation bubbles.

According to a preferred embodiment of the invention, the device comprises a drug reservoir, whose contact area comprises a semipermeable membrane, the passage rate of which depends on the signal intensity of the overall ultrasonic signal. Thus, skin areas which are exposed to high-intensity ultrasound receive a high drug concentration. In other words, in case of a high drug permeation, the drug dosage can be improved. Additionally, the saturation effect can be affected in a positive way. As a membrane, a porous polymeric membrane is preferably used.

The drug reservoir serves not only for containing the drug to be delivered to the patient. Preferably, the drug reservoir is also used for “matching” the acoustic impedances involved. In particular, the drug reservoir is adapted to match the acoustic impedance of the membrane transducer and the acoustic impedance of the patient's skin. If the drug reservoir is positioned directly between the transducer and the patient's skin, the acoustic impedance of the drug reservoir is preferably provided as an average value of the acoustic impedances of transducer and skin. If the transducer has a multilayer or “sandwich” structure, the acoustic impedances of the layers are preferably selected in such a way that there is a smooth transition of the acoustic impedance between transducer and skin.

According to the present invention, the transducer generates a wavefront, which has to be focused on a certain target area on the patient's skin. Well-directed focusing can however only be achieved in the far field, whereas in the near field such a purposeful focusing is not possible. Thus, according to a preferred embodiment of the invention, the distance between the ultrasonic membrane transducer and the target area is at least two times the wavelength of the ultrasonic signals in use. Using low frequencies of about 1 MHz, a distance of 5 mm between the ultrasonic membrane transducer and the target area is sufficient, according to v=f, in which v denotes the acoustic velocity, _{.} denotes the wavelength and f denotes the frequency.

According to yet another preferred embodiment of the invention, the drug reservoir of the device is connected to the housing in such a way that it can easily be replaced. This enables the drug reservoir to be renewed, while the other parts of the device, most notably the ultrasonic membrane transducer with the control unit etc., remain unchanged and can be re-used several times.

The present invention can preferably be used in a medical environment, e.g. for transdermal drug delivery, such as for delivery of peptides, proteins and DNA-based therapeutics, and for pain management.

These and other aspects of the invention will be described in detail hereinafter, by way of example, with reference to the following embodiments and the accompanying drawings; in which:

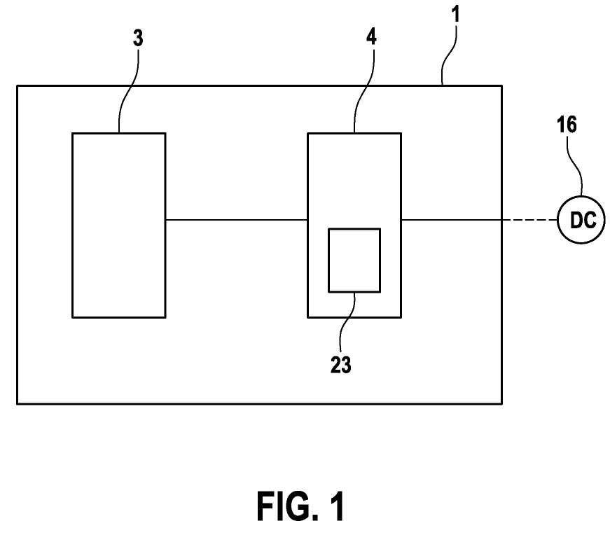

FIG. 1 shows a schematic illustration of an ultrasonic membrane transducer,

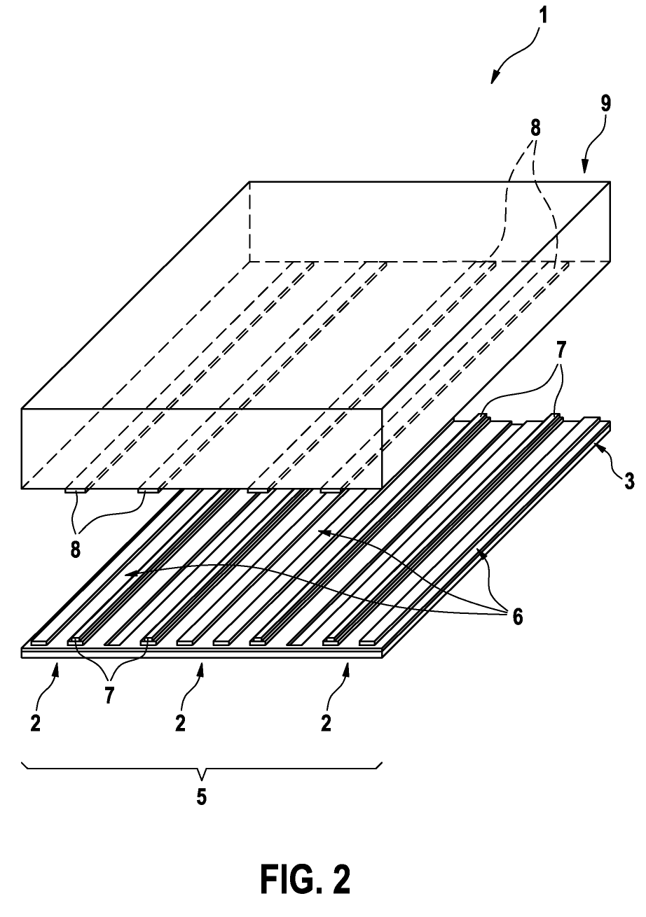

FIG. 2 shows an illustration of a part of the ultrasonic membrane transducer (perspective view),

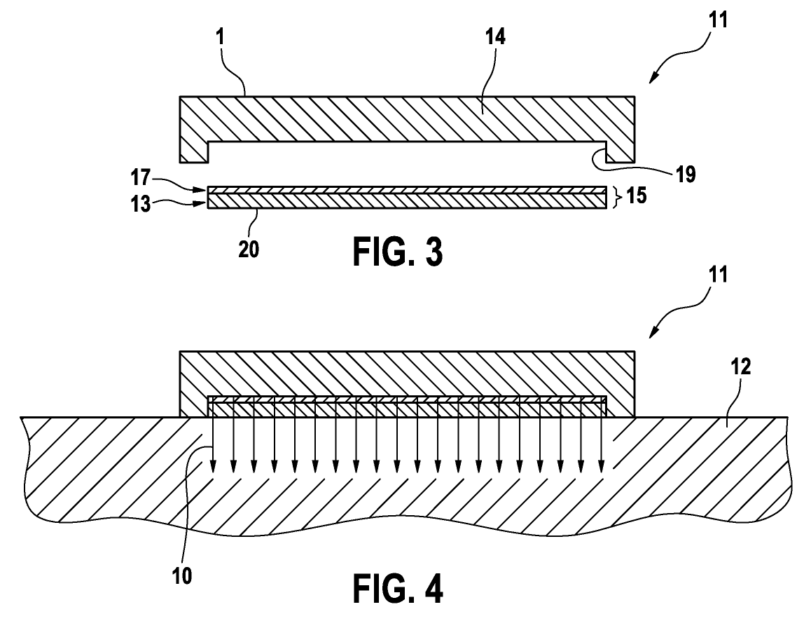

FIG. 3 shows a first illustration of a portable device (side view),

FIG. 4 shows a second illustration of a portable device (side view),

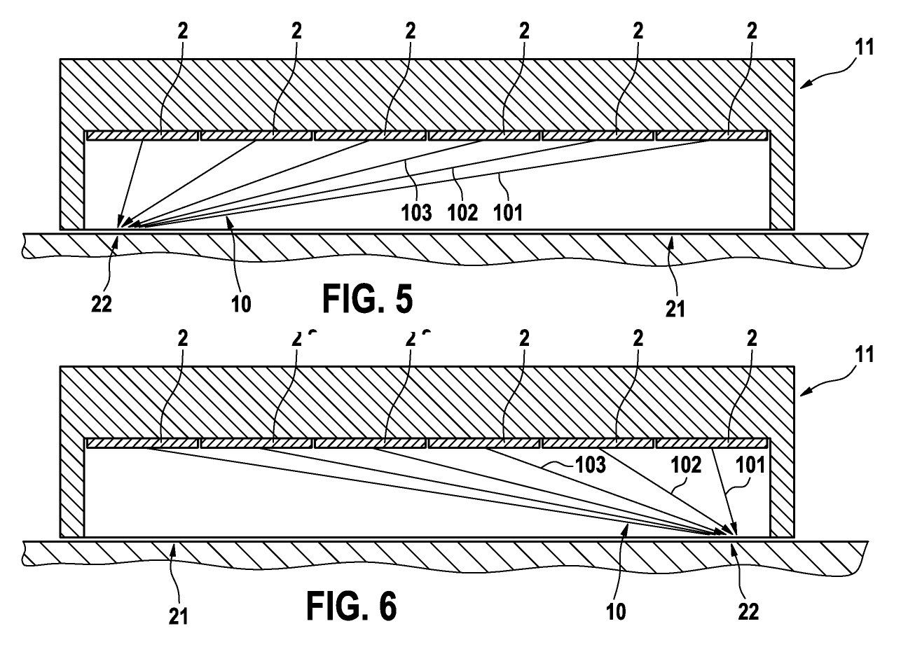

FIG. 5 shows a first illustration of a portable device in use (side view),

FIG. 6 shows a second illustration of a portable device in use (side view), and

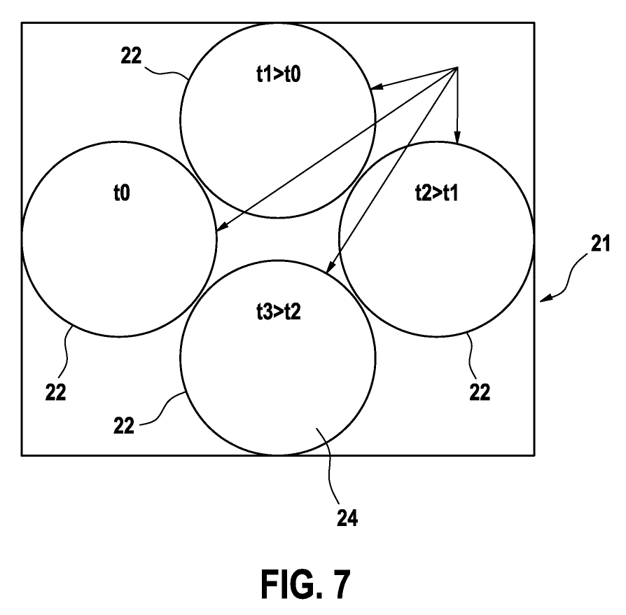

FIG. 7 shows an illustration of a target area (top view).

An ultrasonic membrane transducer 1 according to the present invention comprises a large number, e.g. 64, of transducer elements 2 as parts of a membrane substrate 3, which exhibits a thickness of 1 m to 10 m, and a control unit 4 being electrically coupled to the transducer elements 2, see FIG. 1.

The number of transducer elements 2 form a two-dimensional transducer array

5. In the embodiment as shown in FIG. 2, only one transducer element 2 is depicted completely and two further transducer elements 2 are depicted partly. Ultrasound can be generated e.g. by vibration of a piezoelectric crystal or other electromechanical element by passing an alternating electromagnetic field through the material. In the present embodiment, the transducer elements 2 are composed of a thin film piezoelectric layer 6 and a number of actuation electrodes 7 disposed on the surface of the transducer element 2. The transducer elements 2 are preferably manufactured using low-cost standard semiconductor thin film technology. The actuation electrodes 7 are coupled to the piezoelectric layer 6 for applying an electrical field to the piezoelectric layer 6. As a result, the transducer elements 2 are deflected and an overall ultrasonic signal 10 is generated. The transducer elements 2 work in the frequency range between 0.5 MHz and 50 MHz. The basic technology of such an ultrasonic membrane transducer 1 is known from ultrasonic imaging devices.

The control unit 4 is adapted for separately controlling the application of the electrical field to the piezoelectric layer 6 of each transducer element 2 in such a way that the ultrasonic signals 101, 102, 103, . . . emitted by the transducer elements 2 exhibit phase differences resulting in a focusable overall ultrasonic signal 10 of the transducer array 5. For this purpose, the actuation electrodes 7 of the transducer elements 2 are in electrical contact with contact electrodes 8, which are positioned on a wafer 9 or the like, which is glued or soldered to the membrane substrate 3. The total thickness of the membrane transducer 1 (i.e. membrane substrate 3 together with the wafer 9 carrying the contact electrodes 8) is around 0.5 mm.

A portable device 11 for transdermal drug delivery is shown in FIGS. 3 (disassembled) and 4 (assembled). The device 11 uses the ultrasonic membrane transducer 1 for increasing the permeability of a patient's skin 12 to a chemical substance or drug 13. In addition to the ultrasonic membrane transducer 1, the device 11 further comprises a housing 14 for the ultrasonic membrane transducer 1, and a drug reservoir in form of a pad 15 connected to said housing 14, the pad 15 being positioned adjacent to the ultrasonic membrane transducer 1. Both the actuation electrodes 7 and the control unit 4 are connected to a power supply 16, which is located in the housing 14. Parts of the control unit 4 or the complete control unit 4 can be integrated, at wafer level, on the wafer 9 carrying the contact electrodes 8 in order to miniaturize the device 11.

An ultrasound gel 17 between the drug 13 in the pad 15 and the membrane transducer 1 guarantees that the ultrasonic signals enter the pad 15 nearly without reflection at the boundary 18. The ultrasound gel 17 is preferably provided as part of the pad 15. In other words, drug 13 and gel 17 form a single pad unit. The drug 13 itself is preferably dissolved in the ultrasound gel 17 or a liquid with acoustic impedance nearby the ultrasound gel 17. Preferably, the ultrasonic membrane transducer 1 with its sandwich structure is designed and adapted in such a way that there is an optimum acoustic impedance match between all boundaries or layers, e.g. “matching” layers are used between the transducer array 5 and the patient's skin 12.

The pad 15 of the portable device 11 is connectable to the housing 14 in such a way that it can easily be replaced. For example, the pad is attached to a recess 19 of the housing 14 by means of fasteners (not shown). The device 11 is designed in such a way that the distance between the ultrasonic membrane transducer 1 and the patient's skin 12 is at least two times the wavelength of the ultrasonic signals in use.

The lower surface of the pad 15 comprises a semipermeable membrane 20. The passage rate of the semipermeable membrane 20 depends on the signal intensity of the overall ultrasonic signal 10 received by said membrane 20.

The portable device 1 is adapted to be positioned in such a way that the pad 15 is in close contact with the rectangular skin target area 21, which corresponds to the area of the semipermeable membrane 20. For example, the device 11 comprises attaching means (not shown) for fixing the device to a patient's hand, arm, leg, hip, chest, or the like. The attaching means may comprise, inter alia, tapes, belts, adhesive tapes (plaster).

The operation of the above described ultrasonic membrane transducer 1 within the device 11 is as follows: The control unit 4 applies a drive voltage to each transducer element 2 separately via the corresponding actuation electrodes 7. The drive voltage is applied in such a way that the electrical field to the piezoelectric layer 6 of each transducer element 2 is controlled separately. Thus, the resulting ultrasonic signals 101, 102, 103, . . . generated by each transducer element 2 can be controlled separately. This is done by means of the control unit 4 in such a way that the ultrasonic signals emitted by the transducer elements 2 exhibit phase differences resulting in the focusable overall ultrasonic signal 10 of the transducer array 5. In particular, the position of the focus 22 of the overall ultrasonic signal 10 on the target area 21 can be varied in time, see FIGS. 5 and 6, in which an array of transducer elements is depicted. The ultrasound transducer 1 is working as a phased array in which the phase angles between the transducer elements 2 can be changed continuously or in discrete steps. In FIG. 5, the ultrasonic signals 101, 102, 103, . . . of the number of transducer elements 2 are focused towards the left hand side of the target area 21, and in FIG. 6 the ultrasonic signals 101, 102, 103, . . . of the same number of transducer elements 2 are focused towards the right hand side of the target area 21. The control unit 4 is adapted such that the focus 22 of the overall ultrasonic signal 10 moves across the target area 21 in a defined way.

In FIG. 7, an example of a movement pattern is depicted for an embodiment of the invention in which a two-dimensional transducer array is used. The focus 22 changes position across the target area 21 in time. Different focus positions are shown for tₒ, t₁>tₒ, t₂>t₁ and t₃>t₂. The area 24 of the focus 22 of the overall ultrasonic signal 10 is constant in this embodiment. Another movement pattern would result from a linear transducer array (not shown). In this case, the focus would show the form of a beam, which wanders across the patient's skin from one side of the target area 21 to the other.

The ultrasound signal can be emitted continuously or in a pulsed mode. In the present embodiment, the transducer elements 5 are driven by the control unit 4 in a pulsed mode with a pulse rate between 20,000 and 100,000 pulses per second.

The operation of the control unit 4 is preferably achieved by means of computer software comprising computer instructions adapted for controlling the drive voltage as described above, when the software is executed in a processing unit 23 of the control unit 4. The technical effects necessary according to the invention can thus be realized on the basis of the instructions of the computer program in accordance with the invention. Alternatively, the method of controlling the drive voltages can be implemented in hardware, e.g. using one or more integrated circuits. The processing unit 23 itself may comprise functional modules or units, which are implemented in form of hardware, software or a combination of both.

Recapitulating, the present invention suggests the use of an ultrasonic membrane transducer 1 for increasing the permeability of a patient's skin 12 to drug molecules. For this purpose, the transducer elements 2, which are positioned in form of an array 5, are controlled by means of a dedicated control unit 4 in such a way, that the overall ultrasonic signal 10 of the transducer array 5 can be focused and controllably moved across the target area 21.

It will be evident to those skilled in the art that the invention is not limited to the details of the foregoing illustrative embodiments, and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. It will furthermore be evident that the word “comprising” does not exclude other elements or steps, that the words “a” or “an” do not exclude a plurality, and that a single element, such as a computer system or another unit may fulfil the functions of several means recited in the claims. Any reference signs in the claims shall not be construed as limiting the claim concerned.

### REFERENCE NUMERALS

1 ultrasonic membrane transducer
2 transducer element
3 membrane substrate
4 control unit
5 transducer array
6 piezoelectric layer
7 actuation electrode
8 contact electrode
9 wafer
10 overall ultrasonic signal
11 portable device
12 skin
13 drug
14 housing
15 pad
16 power supply
17 ultrasound gel
18 boundary
19 recess
20 semipermeable membrane
21 target area
22 focus
23 processing unit
24 focus area
101 ultrasonic signals of a single transducer element
102 ultrasonic signals of a single transducer element
103 ultrasonic signals of a single transducer element

1. A device (11) for transdermal drug delivery to a target area (21), e.g. a patient's skin (12), comprising a drug reservoir (15) and an ultrasonic membrane transducer (1) adapted to cooperate with said drug reservoir (15), said ultrasonic membrane transducer (1) comprising
at least two transducer elements (2) forming a transducer array (5), each transducer element (2) having a membrane (6),
a number of electrodes (7) disposed on a surface of each transducer element (2) and coupled to the membrane (6) for applying an electrical field to flex the membrane (6) in order to generate an ultrasonic signal,
a control unit (4) for separately controlling the application of the electrical field to flex the membrane (6) of each transducer element (2) in such a way that the ultrasonic signals (101, 102, 103, . . . ) emitted by the transducer elements (2) exhibit phase differences resulting in a focusable overall ultrasonic signal (10) of the transducer array (5).

2. The device (11) as claimed in claim 1, wherein the membrane (6) comprises a piezoelectric layer and the membrane (6) is flexed by piezoelectric actuation.

3. The device (11) as claimed in claim 1, wherein the membrane (6) is flexed by electrostatic actuation.

4. The device (11) as claimed in claim 1, wherein the control unit (4) of the ultrasonic membrane transducer (1) is adapted to control the application of the electrical field to flex the membrane (6) of each transducer element (2) in such a way that the focus (22) of the overall ultrasonic signal (10) can be varied in time.

5. The device (11) as claimed in claim 1, wherein the control unit (4) of the ultrasonic membrane transducer (1) is adapted to control the application of the electrical field to flex the membrane (6) of each transducer element (2) in such a way that the overall ultrasonic signal (10) is delivered in a pulsed mode, preferably with a pulse rate between 20,000 and 100,000 pulses per second.

6. The device (11) as claimed in claim 1, wherein the drug reservoir (15) comprises a semipermeable membrane (20), whose passage rate depends on the signal intensity of the overall ultrasonic signal (10).

7. The device (11) as claimed in claim 1, wherein the distance between the ultrasonic membrane transducer (1) and the target area (21) is at least two times the wavelength of the ultrasonic signals (101, 102, 103, . . . ) in use.

8. The device (11) as claimed in claim 1, wherein the components of the device (11) are adapted in such a way that the acoustic impedances of said components equal or approximate the acoustic impedance of the patient's skin (12).

9. The device (11) as claimed in claim 1, wherein the drug reservoir (15) is replaceable.

10. The device (11) as claimed in claim 1, comprising a housing (14) for the ultrasonic membrane transducer (1), the drug reservoir (15) being connected to said housing (14), and the drug reservoir (15) being positioned adjacent to the ultrasonic membrane transducer (1), said device (11) being adapted to be positioned in such a way that the drug reservoir (15) is in close contact with the target area (21).

11. A method of operating a device (11) for transdermal drug delivery to a target area (21), e.g. a patient's skin (12), said device (11) comprising a drug reservoir (15) and an ultrasonic membrane transducer (1) adapted to cooperate with said drug reservoir (15), said ultrasonic membrane transducer (1) comprising at least two transducer elements (2) forming a transducer array (5), said method comprising the step of controlling the transducer elements (2) in such a way that the ultrasonic signals (101, 102, 103, . . . ) emitted by the transducer elements (2) exhibit phase differences resulting in a focusable overall ultrasonic signal (10) of the transducer array (5).
